(19) United States
(12) Patent Application Publication
(10) Pub. No.: US 2015/0133685 A1
(43) Pub. Date: May 14, 2015

(54) PROCESS TO PRODUCE A DIENE FROM A LACTONE

(71) Applicant: Agency for Science, Technology and Research, Singapore (SG)

(72) Inventors: Ludger Stubbs, Singapore (SG); Chuanzhao Li, Singapore (SG); Pui Kwan Wong, Singapore (SG); Martin van Meurs, Singapore (SG)

(73) Assignee: Agency for Science, Technology and Research, Singapore (SG)

(21) Appl. No.: 14/388,718

(22) PCT Filed: Mar. 28, 2013
(86) PCT No.: PCT/SG2013/000123

§ 371 Date: Sep. 26, 2014

(30) Foreign Application Priority Data
Mar. 28, 2012 (SG) 201202262-0

(51) Int. Cl. C07C 51/15; C07C 51/00; C07C 5/48; C07C 51/09; C07C 1/207; C07C 5/333

(52) U.S. Cl. CPC C07C 51/15; C07C 1/2078; C07C 5/3335; C07C 5/3332; C07C 5/48; C07C 51/09; C07C 51/00; C07C 2521/12
USPC 562/517; 585/327

(57) ABSTRACT

The invention provides a process for the production of a diene. In the process, a lactone is heated in the presence of a first catalyst system to produce an alkene and carbon dioxide and the alkene is contacted with a second catalyst system to produce an alkyldiene.

### FIELD

This application relates to a process to produce a diene from a lactone.

### PRIORITY

This application claims priority from Singaporean patent application no. 201202262-0, the entire contents of which are incorporated herein by cross-reference.

### BACKGROUND

Concerns for the availability of fossil feedstocks and climate change have led to significant interests in chemicals and polymers derived from biomass. Butadiene is one of the most desired chemicals. It comes mainly as the byproduct of ethylene production from naphtha, Liquefied Petroleum Gas and dehydrogenation of butane. Butadiene is normally used to make styrene butadiene rubber, acrylonitrile-butadiene-styrene, adiponitrile and chloroprene. Among commercial polymers, Nylons and Nylon monomers are exceedingly inefficient in terms of energy and chemical utilization. In this disclosure, we report the invention of a green process for making butadiene and/or adipic acid, a Nylon 66 monomer, from γ-valerolactone. γ-Valerolactone can be derived from hydrogenation of levulinic acid, one of the so-called biobased platform molecules which can be readily obtained from acid-catalyzed decomposition of cellulose or C6 sugars. More generally, the process may be used for production of dienes and/or diacids from lactones.

### SUMMARY OF INVENTION

In a first aspect, there is provided a process for preparing an alkyldiene comprising the steps of:

(a) heating a lactone in the presence of a first catalyst system to produce an alkene and carbon dioxide; and
(b) contacting the alkene with a second catalyst system to produce an alkyldiene.

Advantageously, step (a) is carried out substantially in the absence of water. This may serve to reduce intermediate purification steps and/or to generate less waste streams for treatment and disposal than if water was required for the process. The lactone and/or other components used in step a) (e.g. the first catalyst system, any carrier used) may be dried prior to use in step a).

In one embodiment, the heating of the lactone in step (a) is conducted in a flow reactor. Step (a) may comprise passing the lactone through a bed, optionally a heated bed, of the first catalyst system. The bed may be a packed bed. Optionally, the heating of the lactone in step (a) is carried out at a temperature at or above the normal boiling point of the lactone. Preferably, step (a) is carried out at a temperature of between about 200° C. and about 400° C. or about 220° C. to 360° C. or about 240° C. to 360° C. or about 260° C. to 340° C. or about 280° C. to 330° C. or about 200° C. to 300° C. or about 300° C. to 400° C. or about 250° C. to 300° C. or about 250° C. to 350° C. or about 300° C. to 350° C., e.g. at about 200, 210, 220, 230, 240, 250, 260, 270, 280, 290, 300, 310, 320, 330, 340, 350, 360, 370, 380, 390 or 400° C. In some instances even greater temperatures may be used. The flow in the flow reactor (or through the bed) may be from about 0.2 to about 2 h⁻¹, or about 0.5 to 2, 1 to 2, 0.2 to 1, 0.2 to 0.5, 0.5 to 1.5 or 0.5 to 1 h⁻¹, e.g. about 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2 h⁻¹, although other flow rates may be used in particular cases. The unit h⁻¹ will be recognised to be a volume independent unit, which may be considered equivalent, for example, to litres reagent per hour per litre of bed volume.

In one embodiment, step (a) is carried out at a pressure of between about 0.5 bar and about 50 bar, or between about 0.5 and 25, 25 and 50, 10 and 40, 0.5 and 20, 0.5 and 10, 0.5 and 5, 0.5 and 2, 0.5 and 1, 1 and 50, 10 and 50, 20 and 50, 10 and 50, 10 and 20, 5 and 20 or 20 and 30 bar, e.g. about 0.5, 1, 1.5, 2, 2.5, 3, 3.5, 4, 4.5, 5, 6, 7, 8, 9, 10, 15, 20, 25, 30, 35, 40, 45 or 50 bar. In some instances even higher pressures may be used. The pressure may be such that, at the bed temperature, the lactone is a vapour.

In one embodiment, the first catalyst system comprises an acidic catalyst. The first catalyst system, e.g. the acidic catalyst, may be a heterogeneous solid catalyst. The acidic catalyst may also be a homogeneous catalyst. The acidic catalyst may be selected from a group consisting of alumina, silica, a zeolite, a clay, sulphuric acid, p-toluenesulfonic acid and methanesulfonic acid. Mixtures of these may also be used. The second catalyst system may comprise a metal based catalyst. It may comprise a tin catalyst, such as tin phosphate catalyst. The tin phosphate catalyst may be lithium promoted. The second catalyst system may comprise a mixed metal catalyst. One of said metals may be molybdenum. Another (or the other) may be cobalt or zinc. The mixed metal may be used in conjunction with oxygen. It may be activated, e.g. activated by chromium. It may be combined with a modifier, e.g. an alkali oxide or hydroxide modifier. It may be supported or unsupported. It may be supported on a mineral support e.g. alumina, silica etc. It may be calcined before use. The mixed metal may be vanadium/iron or vanadium/nickel. The second catalyst system may be zirconia based or iron oxide based. It may comprise some other dehydrogenation catalyst. The second catalyst system may be a mixed metal oxide. It may be bismuth-molybdenum oxide, iron-zinc oxide, transition metal promoted magnesium oxide or vanadium phosphorus oxide.

In one embodiment, the alkene of step (a) comprises a plurality of isomers.

In one embodiment, the process of step (b) comprises the step of direct dehydrogenation of the alkene to produce the alkyldiene.

In another embodiment, the process of step (b) comprises the step of oxidative dehydrogenation of the alkene to produce the alkyldiene. This reaction is known, see for example Kirk-Othmer Encyclopedia of Chemical Technology, Volume 4, p 365-392, 5^{th} Ed., 2004, John Wiley & Sons section on Butadiene (authors H. N. Sun and J. P. Wristers) and reference therein: “Normal butenes can be oxidatively dehydrogenated to butadiene in the presence of high concentration of steam with fairly high selectivity. The conversion is no longer limited by thermodynamics because of the oxidation of hydrogen to water. Reaction temperature is below 600° C. to minimize over oxidation. Pressure is ˜34-103 kPa (5-15 psi).” The reaction may be conducted, for example, at a temperature of about 400 to about 600° C., or about 400 to 500, 500 to 600 or 450 to 550° C., e.g. about 400, 450, 500, 500 or 600° C. It may be conducted at a pressure of about 30 to about 110 kPa, or about 50 to 110, 80 to 110, 30 to 100, 30 to 70, 30 to 50 or 50 to 100 kPa, e.g. about 30, 40, 50, 60, 70, 80, 90, 100 or 110 kPa.

In another embodiment, a portion of the lactone may be unreacted after step (a), whereby a reaction composition comprising unreacted lactone and alkene is produced in step (a).

In another embodiment, the process further comprises the step of:

(a1) separating part, or substantially all, of the unreacted lactone from the alkene.

In another embodiment, the process further comprises the step of:

(a2) recycling the separated unreacted lactone from step (a1) to step (a).

In one embodiment, a portion of the alkene may be unreacted after step (b), and a reaction composition comprising unreacted alkene and alkyldiene is produced in step (b).

In another embodiment, the process further comprises the step of:

(b1) separating part or substantially all of the unreacted alkene from the alkyldiene.

In another embodiment, the process further comprises the step of:

(b2) recycling the separated unreacted alkene from step (b1) to step (b).

In one embodiment, heating the lactone in the presence of the first catalyst system in step (a) further produces an alkenoic acid. The alkenoic acid may comprise a plurality of isomers. These isomers may include a substituted acrylic acid.

In another embodiment, the process further comprises the step of:

(a3) separating part or substantially all of the alkenoic acid from the alkene.

In another embodiment, the process further comprises the step of:

(c) contacting the alkenoic acid with carbon monoxide, water and a third catalyst system to produce a reaction composition comprising the third catalyst system and a dicarboxylic acid.

In another embodiment, step (c) is carried out substantially in the absence of oxygen. It may be conducted in an atmosphere containing less than 0.1% v/v oxygen, or less than about 0.01, 0.001, 0.0001 or 0.00001% oxygen v/v. It may be conducted under an atmosphere of carbon monoxide, nitrogen, helium, neon, argon or a mixture of any two or more of these, or under some other anoxic atmosphere.

In another embodiment, step (c) is carried out at a temperature of between about 50° C. and about 150° C. Step (c) may be carried out at a temperature of between about 50 and 100, 100 and 150, 70 and 100, 100 and 130, 80 and 120, 85 and 115, 90 and 110, 95 and 110, 80 and 100, 100 and 120, 100 and 105 90 and 100 or 100 and 110° C., e.g. about 50, 60, 70, 80, 90, 100, 110, 120, 130, 140 or 150° C., or at some other temperature.

In one embodiment, step (c) is carried out at a pressure of between about 1 bar and about 80 bar, or between about 1 and 40, 1 and 20, 1 and 10, 1 and 5, 10 and 80, 20 and 80, 40 and 80, 10 and 50, 20 and 60, 30 and 70 or 40 and 60 bar, e.g. about 1, 2, 3, 4, 5, 6, 7, 8, 9, 10, 15, 20, 25, 30, 35, 40, 45, 50, 55, 60, 65, 70, 75 or 80 bar, or at some higher pressure.

In one embodiment, the third catalyst system comprises a palladium catalyst.

The palladium catalyst may have the formula (I):

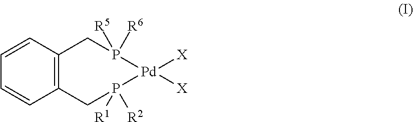

wherein X is a ligand and each of R¹, R², R⁵ and R⁶ is independently an optionally substituted hydrocarbon group. The aromatic ring of (I) may be substituted (other than as shown above) or may be unsubstituted (other than as shown above). R¹, R², R⁵ and R⁶ may each independently represent a tertiary alkyl, cycloalkyl, heterocyclyl or aryl. R1, R², R⁵ and R⁶ may each independently represent tertiary butyl. In a particular embodiment, R1, R², R⁵ and R⁶ are each tertiary butyl and X is CH₃SO₃⁻.

In one embodiment, the term X in formula (I) is derived from an acid, e.g. a sulfonic acid, sulphuric acid, phosphorous acid or carboxylic acid.

In one embodiment, the palladium catalyst is prepared by combining a palladium compound, a bidentate diphosphine and an acid.

In some embodiments, the palladium catalyst is prepared in situ.

In some embodiments, the palladium compound is selected from the group consisting of palladium carboxylates and palladium(0) compounds. The palladium compound may be palladium acetate.

In another embodiment, the acid for preparing the palladium catalyst is selected from the group consisting of sulfonic acids, sulphuric acids, phosphorous acids and carboxylic acids.

In another embodiment, the acid is a C₁-C₁₀ aliphatic acid. The C₁ to C₁₀ chain may be straight chain or branched or may be cyclic or may be a combination of these. It may be C1 to C6, C1 to C3, C2 to C10, C6 to C10 or C3 to C6, e.g. C1, C2, C3, C4, C5, C6, C7, C8, C9 or C10.

In another embodiment, the acid is selected from the group consisting of methanesulfonic acid, triflic acid, trifluoroacetic acid and p-toluenesulfonic acid. The acid may for example be methane sulfonic acid.

In another embodiment, the bidentate diphosphine for the palladium catalyst preparation is 1,2-bis[di(t-butyl)phosphinomethyl]benzene.

In another embodiment, the acid is present in molar excess relative to the palladium compound. The molar ratio of the acid to the palladium compound may be from about 2:1 to about 10000:1 or from about 3:1 to 10000:1, 5:1 to 10000:1, 10:1:10000:1, 100:1 to 10000:1, 1000:1 to 10000:1, 2:1 to 1000:1, 2:1 to 100:1, 2:1 to 10:1, 10:1 to 100:1, 10:1 to 1000:1, 50:1 to 500:1, 5:1 to 5000:1, 5:1 to 300:1 or 10:1 to 100:1. The molar ratio of the acid to the palladium compound may be about 2:1, 3:1, 4:1, 5:1, 10:1, 20:1, 50:1, 100:1, 200:1, 500:1, 1000:1, 2000:1, 5000:1 or 10000:1. It may be from about 5:1 to about 300:1.

In another embodiment, a portion of the lactone is unreacted after step (a), producing a reaction composition comprising unreacted lactone and alkene and part or substantially all of the unreacted lactone is present in step (c) producing a reaction composition comprising unreacted lactone and dicarboxylic acid.

In another embodiment, the process further comprises the step of:

(c1) separating part or substantially all the unreacted lactone from the dicarboxylic acid.

In another embodiment, the process further comprises the step of:

(c2) recycling the separated unreacted lactone from step (c1) to step (a).

In another embodiment, a portion of the alkenoic acid is unreacted after step (c), producing a reaction composition comprising unreacted alkenoic acid and dicarboxylic acid from step (c).

In another embodiment, the process further comprises the step of:

(c3) separating part or substantially all of the unreacted alkenoic acid from the dicarboxylic acid.

In another embodiment, the process further comprises the step of:

(c4) recycling the separated unreacted alkenoic acid from step (c3) to step (c).

In another embodiment, the process further comprises the step of:

(c5) separating the dicarboxylic acid from the remainder of the reaction composition to produce a first portion comprising the dicarboxylic acid and a second portion comprising the third catalyst system. The separating may comprise reducing the temperature to produce the first portion comprising the dicarboxylic acid and substantially none of the third catalyst system, and a second portion comprising the third catalyst system. The first portion may be a solid portion. It may consist essentially of the dicarboxylic acid (e.g. greater than about 95, 96, 907, 98 or 99% w/w). The separating may comprise crystallising the dicarboxylic acid.

In another embodiment, the first portion comprises substantially none of the third catalyst system.

In another embodiment, step (c5) further comprises the step of reducing the temperature of the reaction composition such that the dicarboxylic acid crystallises. The reduction in temperature may be by at least about 5 Celsius degrees, or by at least about 10, 50, 20, 25, 30, 35, 40, 45 or 50 Celsius degrees. In some instances a solvent is present, either from step (c) or separately added after step (c). The solvent may be such that the dicarboxylic acid is recrystallisable therefrom. It may be such that the dicarboxylic acid is substantially more soluble therein at high temperature than at low temperature. In this context, the difference between high and low temperatures may be at least about 5 Celsius degrees, or at least about 10, 50, 20, 25, 30, 35, 40, 45 or 50 Celsius degrees.

In another embodiment, the process further comprises the step of:

(c6) recycling the second portion, comprising the third catalyst system, to step (c).

In one embodiment, the lactone is γ-valerolactone, the alkene is butene and the alkyldiene is 1,3-butadiene. The lactone may be a bio-based lactone. It may be bio-based valerolactone. The process may comprise the step of preparing the γ-valerolactone. This may for example be by hydrogenation of levulinic acid. The process may further comprise the step of preparing the levulinic acid, e.g. by acid catalysed hydrolysis of cellulose. The process may further comprise the step of preparing the cellulose, e.g. by cracking lignocellulose.

In one embodiment, the butene comprises 1-butene and 2-butene.

In one embodiment, the lactone is γ-valerolactone, whereby the alkene is butene, the alkyldiene is 1,3-butadiene, the alkenoic acid is pentenoic acid and the dicarboxylic acid is adipic acid.

In one embodiment, the pentenoic acid comprises a mixture of at least two of 2-pentenoic acid, 3-pentenoic acid and 4-pentenoic acid.

In one embodiment, the γ-valerolactone is prepared by hydrogenation of levulinic acid.

In one embodiment, the levulinic acid is prepared by acid catalysed hydrolysis of cellulose.

In one embodiment, the cellulose is prepared by cracking lignocellulose.

In a second aspect, there is provided a process for preparing a dicarboxylic acid comprising the steps of:

(a) heating a lactone in the presence of a first catalyst system to produce an alkenoic acid and carbon dioxide; and
(b) contacting the alkenoic acid with carbon monoxide, water and a second catalyst system to produce a reaction composition comprising the second catalyst system and the dicarboxylic acid.

In one embodiment, heating the lactone in the presence of the first catalyst system in step (a) further produces an alkene.

The process may comprise the step of (c) contacting the alkene with a third catalyst system to produce an alkyldiene.

Advantageously, step (a) is carried out substantially in the absence of water, reducing intermediate purification steps and generating less waste streams for treatment and disposal than if water was required for the process.

In one embodiment, the heating of the lactone in step (a) is conducted in a flow reactor. Optionally, the heating of the lactone in step (a) is carried out at a temperature at or above the normal boiling point of the lactone. Preferably, step (a) is carried out at a temperature of between 200° C. and 400° C. or 220° C. and 360° C. or 240° C. and 360° C. or 260° C. and 340° C. or 280° C. and 330° C.

In one embodiment, step (a) is carried out at a pressure of between about 0.5 bar and 50 bar, or 0.5 bar to 25 bar, or 25 bar to 50 bar, or 10 bar to 40 bar.

In one embodiment, the first catalyst system comprises an acidic catalyst. The acidic catalyst may be a heterogeneous solid catalyst. The acidic catalyst may also be a homogeneous catalyst. The acidic catalyst may be selected from a group consisting of alumina, silica, a zeolite, a clay, sulphuric acid, p-toluenesulfonic acid and methanesulfonic acid.

In another embodiment, a portion of the lactone may be unreacted after step (a), whereby a reaction composition comprising unreacted lactone and alkenoic acid is produced in step (a).

In another embodiment, the process further comprises the step of:

(a1) separating part, or substantially all, of the unreacted lactone from the alkenoic acid.

In another embodiment, the process further comprises the step of:

(a2) recycling the separated unreacted lactone from step (a1) to step (a).

In another embodiment, the process further comprises the step of:

(a3) separating part or substantially all of the alkenoic acid from the alkene.

In one embodiment, the alkenoic acid of step (a) comprises a plurality of isomers.

In another embodiment, step (b) is carried out substantially in the absence of oxygen.

In another embodiment, step (b) is carried out at a temperature of between 50° C. and 150° C. More preferably, step (b) is carried out at a temperature of between 80° C. and 120° C., or 85° C. to 115° C., or 90° C. to 110° C., or 95° C. to 110° C., or 80° C. to 100° C. or 100° C. to 120° C. or 100° C. to 105° C.

In one embodiment, step (b) is carried out at a pressure of between 1 bar and 80 bar, or between 1 bar and 40 bar, or between 1 bar and 20 bar, or between 40 bar and 80 bar, or between 10 bar and 50 bar, or between 20 bar and 60 bar, or between 30 bar and 70 bar.

In one embodiment, the second catalyst system comprises a palladium catalyst.

The palladium catalyst may have the formula (I):

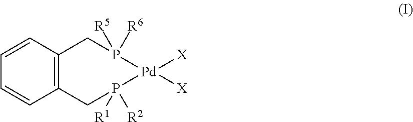

wherein X is a ligand and each of R¹, R², R⁵ and R⁶ is independently an optionally substituted hydrocarbon group. Preferably, R¹, R², R⁵ and R⁶ each independently represent a tertiary alkyl, cycloalkyl, heterocyclyl or aryl. Most preferably, R1, R², R⁵ and R⁶ each independently represent tertiary butyl.

In one embodiment, the term X in formula (I) is derived from an acid.

In one embodiment, the palladium catalyst is prepared by combining a palladium compound, a bidentate diphosphine and an acid.

In another embodiment, the palladium catalyst is prepared in situ.

In another embodiment, the palladium compound is selected from the group consisting of palladium carboxylates and palladium(0) compounds. Preferably the palladium compound is palladium acetate.

In another embodiment, the acid for preparing the palladium catalyst is selected from the group consisting of sulfonic acids, sulphuric acids, phosphorous acids and carboxylic acids.

In another embodiment, the acid is a C₁-C₁₀ aliphatic acid.

In another embodiment, the acid is selected from the group consisting of methanesulfonic acid, triflic acid, trifluoroacetic acid and p-toluenesulfonic acid. Preferably, the acid is methane sulfonic acid.

In another embodiment, the bidentate diphosphine for the palladium catalyst preparation is 1,2-bis[di(t-butyl)phosphinomethyl]benzene.

In another embodiment, the acid is present in molar excess relative to the palladium compound. Preferably the molar ratio of the acid to the palladium compound is from 2:1 to 10000:1 or from 5:1 to 5000:1 or from 5:1 to 300:1. Most preferably the molar ratio of the acid to the palladium compound is from 5:1 to 300:1.

In another embodiment, a portion of the lactone is unreacted after step (a), producing a reaction composition comprising unreacted lactone and alkenoic acid and part or substantially all of the unreacted lactone is present in step (b) producing a reaction composition comprising unreacted lactone and dicarboxylic acid.

In another embodiment, the process further comprises the step of:

(b1) separating part or substantially all the unreacted lactone from the dicarboxylic acid.

In another embodiment, the process further comprises the step of:

(b2) recycling the separated unreacted lactone from step (b1) to step (a).

In another embodiment, a portion of the alkenoic acid is unreacted after step (b), producing a reaction composition comprising unreacted alkenoic acid and dicarboxylic acid from step (b).

In another embodiment, the process further comprises the step of:

(b3) separating part or substantially all of the unreacted alkenoic acid from the dicarboxylic acid.

In another embodiment, the process further comprises the step of:

(b4) recycling the separated unreacted alkenoic acid from step (b3) to step (b).

In another embodiment, the process further comprises the step of:

(b5) separating the dicarboxylic acid from the remainder of the reaction composition to produce a first portion comprising the dicarboxylic acid and a second portion comprising the second catalyst system.

In another embodiment, the first portion comprises substantially none of the second catalyst system.

In another embodiment, step (b5) further comprises the step of reducing the temperature of the reaction composition such that the dicarboxylic acid crystallises.

In another embodiment, the process further comprises the step of:

(b6) recycling the second portion, comprising the second catalyst system, to step (b).

In one embodiment, the alkene of step (a) comprises a plurality of isomers.

In one embodiment, the process of step (c) comprises the step of direct dehydrogenation of the alkene to produce the alkyldiene.

In another embodiment, the process of step (c) comprises the step of oxidative dehydrogenation of the alkene to produce the alkyldiene.

In one embodiment, a portion of the alkene may be unreacted after step (c), and a reaction composition comprising unreacted alkene and alkyldiene is produced in step (c).

In another embodiment, the process further comprises the step of:

(c1) separating part or substantially all of the unreacted alkene from the alkyldiene.

In another embodiment, the process further comprises the step of:

(c2) recycling the separated unreacted alkene from step (c1) to step (c).

In one embodiment, the lactone is γ-valerolactone, the alkene is butene and the alkyldiene is 1,3-butadiene.

In one embodiment, the butene comprises 1-butene and 2-butene.

In one embodiment, the lactone is γ-valerolactone, whereby the alkene is butene, the alkyldiene is 1,3-butadiene, the alkenoic acid is pentenoic acid and the dicarboxylic acid is adipic acid.

In one embodiment, the pentenoic acid comprises 2-pentenoic acid, 3-pentenoic acid and 4-pentenoic acid.

In one embodiment, the γ-valerolactone is prepared by hydrogenation of levulinic acid.

In one embodiment, the levulinic acid is prepared by acid catalysed hydrolysis of cellulose.

In one embodiment, the cellulose is prepared by cracking lignocellulose.

Also disclosed is a process for preparing an alkyldiene and a dicarboxylic acid comprising the steps of:

(a) heating a lactone in the presence of a first catalyst system to produce an alkenoic acid, an alkene and carbon dioxide; and
(b) contacting the alkene with a second catalyst system to produce an alkyldiene.
(c) contacting the alkenoic acid with carbon monoxide, water and a third catalyst system to produce a reaction composition comprising the third catalyst system and the dicarboxylic acid.

Advantageously, step (a) is carried out substantially in the absence of water, reducing intermediate purification steps and generating less waste streams for treatment and disposal than if water was required for the process.

In one embodiment, the heating of the lactone in step (a) is conducted in a flow reactor. Optionally, the heating of the lactone in step (a) is carried out at a temperature at or above the normal boiling point of the lactone. Preferably, step (a) is carried out at a temperature of between 200° C. and 400° C. or 220° C. to 360° C. or 240° C. to 360° C. or 260° C. to 340° C. or 280° C. to 330° C.

In one embodiment, step (a) is carried out at a pressure of between about 0.5 bar and 50 bar, or 0.5 bar to 25 bar, or 25 bar to 50 bar, or 10 bar to 40 bar.

In one embodiment, the first catalyst system comprises an acidic catalyst. The acidic catalyst may be a heterogeneous solid catalyst. The acidic catalyst may also be a homogeneous catalyst. The acidic catalyst may be selected from a group consisting of alumina, silica, a zeolite, a clay, sulphuric acid, p-toluenesulfonic acid and methanesulfonic acid.

In another embodiment, a portion of the lactone may be unreacted after step (a), whereby a reaction composition comprising unreacted lactone, alkenoic acid and alkene is produced in step (a).

In another embodiment, the process further comprises the step of:

(a1) separating part, or substantially all, of the unreacted lactone from the alkenoic acid and alkene.

In another embodiment, the process further comprises the step of:

(a2) recycling the separated unreacted lactone from step (a1) to step (a).

In another embodiment, the process further comprises the step of:

(a3) separating part or substantially all of the alkenoic acid from the alkene.

In one embodiment, the alkene of step (a) comprises a plurality of isomers.

In one embodiment, the process of step (b) comprises the step of direct dehydrogenation of the alkene to produce the alkyldiene.

In another embodiment, the process of step (b) comprises the step of oxidative dehydrogenation of the alkene to produce the alkyldiene.

In one embodiment, a portion of the alkene may be unreacted after step (b), and a reaction composition comprising unreacted alkene and alkyldiene is produced in step (b).

In another embodiment, the process further comprises the step of:

(b1) separating part or substantially all of the unreacted alkene from the alkyldiene.

In another embodiment, the process further comprises the step of:

(b2) recycling the separated unreacted alkene from step (b1) to step (b).

In another embodiment, the alkenoic acid may comprise a plurality of isomers.

In another embodiment, step (c) is carried out substantially in the absence of oxygen.

In another embodiment, step (c) is carried out at a temperature of between 50° C. and 150° C. More preferably, step (c) is carried out at a temperature of between 80° C. and 120° C., or 85° C. to 115° C., or 90° C. to 110° C., or 95° C. to 110° C., or 80° C. to 100° C. or 100° C. to 120° C. or 100° C. to 105° C.

In one embodiment, step (c) is carried out at a pressure of between 1 bar and 80 bar, or between 1 bar and 40 bar, or between 1 bar and 20 bar, or between 40 bar and 80 bar, or between 10 bar and 50 bar, or between 20 bar and 60 bar or between 30 bar and 70 bar.

In one embodiment, the third catalyst system comprises a palladium catalyst.

The palladium catalyst may have the formula (I):

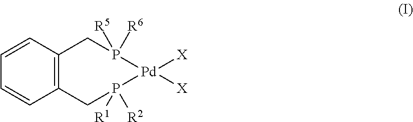

wherein X is a ligand and each of R¹, R², R⁵ and R⁶ is independently an optionally substituted hydrocarbon group. Preferably, R¹, R², R⁵ and R⁶ each independently represent a tertiary alkyl, cycloalkyl, heterocyclyl or aryl. Most preferably, R¹, R², R⁵ and R⁶ each independently represent tertiary butyl.

In one embodiment, the term X in formula (I) is derived from an acid.

In one embodiment, the palladium catalyst is prepared by combining a palladium compound, a bidentate diphosphine and an acid.

In another embodiment, the palladium catalyst is prepared in situ.

In another embodiment, the palladium compound is selected from the group consisting of palladium carboxylates and palladium(0) compounds. Preferably the palladium compound is palladium acetate.

In another embodiment, the acid for preparing the palladium catalyst is selected from the group consisting of sulfonic acids, sulphuric acids, phosphorous acids and carboxylic acids.

In another embodiment, the acid is a C₁-C₁₀ aliphatic acid.

In another embodiment, the acid is selected from the group consisting of methanesulfonic acid, triflic acid, trifluoroacetic acid and p-toluenesulfonic acid. Preferably, the acid is methane sulfonic acid.

In another embodiment, the bidentate diphosphine for the palladium catalyst preparation is 1,2-bis[di(t-butyl)phosphinomethyl]benzene.

In another embodiment, the acid is present in molar excess relative to the palladium compound. Preferably the molar ratio of the acid to the palladium compound is from 2:1 to 10000:1 or from 5:1 to 5000:1 or from 5:1 to 300:1. Most preferably the molar ratio of the acid to the palladium compound is from 5:1 to 300:1.

In another embodiment, a portion of the lactone is unreacted after step (a), producing a reaction composition comprising unreacted lactone, alkenoic acid and alkene and part or substantially all of the unreacted lactone is present in step (c) producing a reaction composition comprising unreacted lactone and dicarboxylic acid.

In another embodiment, the process further comprises the step of:

(c1) separating part or substantially all the unreacted lactone from the dicarboxylic acid.

In another embodiment, the process further comprises the step of:

(c2) recycling the separated unreacted lactone from step (c1) to step (a).

In another embodiment, a portion of the alkenoic acid is unreacted after step (c), producing a reaction composition comprising unreacted alkenoic acid and dicarboxylic acid from step (c).

In another embodiment, the process further comprises the step of:

(c3) separating part or substantially all of the unreacted alkenoic acid from the dicarboxylic acid.

In another embodiment, the process further comprises the step of:

(c4) recycling the separated unreacted alkenoic acid from step (c3) to step (c).

In another embodiment, the process further comprises the step of:

(c5) separating the dicarboxylic acid from the remainder of the reaction composition to produce a first portion comprising the dicarboxylic acid and a second portion comprising the third catalyst system.

In another embodiment, the first portion comprises substantially none of the third catalyst system.

In another embodiment, step (c5) further comprises the step of reducing the temperature of the reaction composition such that the dicarboxylic acid crystallises.

In another embodiment, the process further comprises the step of:

(c6) recycling the second portion, comprising the third catalyst system, to step (c).

In one embodiment, the lactone is γ-valerolactone, the alkene is butene and the alkyldiene is 1,3-butadiene.

In one embodiment, the butene comprises 1-butene and 2-butene.

In one embodiment, the lactone is γ-valerolactone, whereby the alkene is butene, the alkyldiene is 1,3-butadiene, the alkenoic acid is pentenoic acid and the dicarboxylic acid is adipic acid.

In one embodiment, the pentenoic acid comprises 2-pentenoic acid, 3-pentenoic acid and 4-pentenoic acid.

In one embodiment, the γ-valerolactone is prepared by hydrogenation of levulinic acid.

In one embodiment, the levulinic acid is prepared by acid catalysed hydrolysis of cellulose.

In one embodiment, the cellulose is prepared by cracking lignocellulose.

In third aspect, there is provided a process to produce an alkyldiene and a dicarboxylic acid, the process comprising the steps of:

(a) heating a lactone in the presence of a first catalyst system to produce an alkene, an alkenoic acid and carbon dioxide; and
(b) contacting the alkene of step (a) with a second catalyst system to produce the alkyldiene,
(c) contacting the alkenoic acid of step (a) with carbon monoxide and water and a third catalyst system to produce the dicarboxylic acid.

The process may comprise the step of (d) removing the alkyldiene from the carbon monoxide and water before step (c). It should be noted that butadiene may be used a co-feed for step (c). It may slow down the carbonylation reaction or may place different requirements on the Pd catalyst due to pi-allyl formation. Also, small amounts of butene from step (b) may end up in step (c) with the carbon monoxide, however this is unlikely to cause a problem: it is likely that they would be carbonylated to valeric acid, a compound that is formed in small amounts during both step (a) and (c) in any event.

The process may comprise the step of (e) removing the dicarboxylic acid from the third catalyst system after step (c).

Advantageously, step (a) is carried out substantially in the absence of water, reducing intermediate purification steps and generating less waste streams for treatment and disposal than if water was required for the process.

In one embodiment, the heating of the lactone in step (a) is conducted in a flow reactor. Optionally, the heating of the lactone in step (a) is carried out at a temperature at or above the normal boiling point of the lactone. Preferably, step (a) is carried out at a temperature of between 200° C. and 400° C. or 220° C. to 360° C. or 240° C. to 360° C. or 260° C. to 340° C. or 280° C. to 330° C.

In one embodiment, step (a) is carried out at a pressure of between about 0.5 bar and 50 bar, or 0.5 bar to 25 bar, or 25 bar to 50 bar, or 10 bar to 40 bar.

In one embodiment, the first catalyst system comprises an acidic catalyst. The acidic catalyst may be a heterogeneous solid catalyst. The acidic catalyst may also be a homogeneous catalyst. The acidic catalyst may be selected from a group consisting of alumina, silica, a zeolite, a clay, sulphuric acid, p-toluenesulfonic acid and methanesulfonic acid.

In one embodiment, the alkene of step (a) comprises a plurality of isomers.

In another embodiment, the process of step (b) comprises the step of oxidative dehydrogenation of the alkene to produce the alkyldiene.

In another embodiment, a portion of the lactone may be unreacted after step (a), whereby a reaction composition comprising unreacted lactone and alkene is produced in step (a).

In, another embodiment, the process further comprises the step of:

(a1) separating part, or substantially all, of the unreacted lactone from the alkene.

In another embodiment, the process further comprises the step of:

(a2) recycling the separated unreacted lactone from step (a1) to step (a).

In one embodiment, a portion of the alkene may be unreacted after step (b), and a reaction composition comprising unreacted alkene and alkyldiene is produced in step (b).

In another embodiment, the process further comprises the step of:

(b1) separating part or substantially all of the unreacted alkene from the alkyldiene.

In another embodiment, the process further comprises the step of:

(b2) recycling the separated unreacted alkene from step (b1) to step (b).

In one embodiment, the alkenoic acid of step (a) may comprise a plurality of isomers.

In another embodiment, the process further comprises the step of:

(a3) separating part or substantially all of the alkenoic acid from the alkene.

In another embodiment, step (c) is carried out substantially in the absence of oxygen.

In another embodiment, step (c) is carried out at a temperature of between 50° C. and 150° C. More preferably, step (c) is carried out at a temperature of between 80° C. and 120° C. or 85° C. to 115° C. or 90° C. to 110° C. or 95° C. to 110° C. or 100° C. to 105° C.

In one embodiment, step (c) is carried out at a pressure of between 1 bar and 80 bar or between 1 bar and 40 bar or between 1 bar and 20 bar, or between 40 bar and 80 bar, or between 10 bar and 50 bar, or between 20 bar and 60 bar or between 30 bar and 70 bar.

In one embodiment, the third catalyst system comprises a palladium catalyst.

The palladium catalyst may have the formula (I):

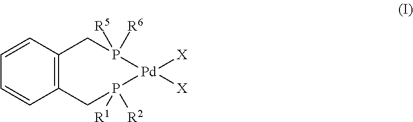

wherein X is a ligand and each of R¹, R², R⁵ and R⁶ is independently an optionally substituted hydrocarbon group. Preferably, R¹, R², R⁵ and R⁶ each independently represent a tertiary alkyl, cycloalkyl, heterocyclyl or aryl. Most preferably, R¹, R², R⁵ and R⁶ each independently represent tertiary butyl.

In one embodiment, the term X in formula (I) is derived from an acid.

In one embodiment, the palladium catalyst is prepared by combining a palladium compound, a bidentate diphosphine and an acid.

In another embodiment, the palladium catalyst is prepared in situ.

In another embodiment, the palladium compound is selected from the group consisting of palladium carboxylates and palladium(0) compounds. Preferably the palladium compound is palladium acetate.

In another embodiment, the acid for preparing the palladium catalyst is selected from the group consisting of sulfonic acids, sulphuric acids, phosphorous acids and carboxylic acids.

In another embodiment, the acid is a C₁-C₁₀ aliphatic acid.

In another embodiment, the acid is selected from the group consisting of methanesulfonic acid, triflic acid, trifluoroacetic acid and p-toluenesulfonic acid. Preferably, the acid is methane sulfonic acid.

In another embodiment, the bidentate diphosphine for the palladium catalyst preparation is 1,2-bis[di(t-butyl)phosphinomethyl]benzene.

In another embodiment, the acid is present in molar excess relative to the palladium compound. Preferably the molar ratio of the acid to the palladium compound is from 2:1 to 10000:1 or from 5:1 to 5000:1 or from 5:1 to 300:1. Most preferably the molar ratio of the acid to the palladium compound is from 5:1 to 300:1.

In another embodiment, a portion of the lactone is unreacted after step (a), producing a reaction composition comprising unreacted lactone, alkenoic acid and alkene and part or substantially all of the unreacted lactone is present in step (c) producing a reaction composition comprising unreacted lactone and dicarboxylic acid.

In a fourth aspect, there is provided an alkyldiene prepared in accordance with the process as defined above.

In one embodiment, the alkyldiene has a purity of greater than 99%.

In one embodiment, the alkyldiene is 1,3-butadiene.

In a fifth aspect, there is provided use of the 1,3-butadiene produced by the process as defined above in the manufacture of styrene butadiene rubber, acrylonitrile-butadiene-styrene, adiponitrile or chloroprene.

In a sixth aspect, there is provided a dicarboxylic acid prepared in accordance with the process as defined above.

In one embodiment, the dicarboxylic acid has a purity of greater than 99%.

In one embodiment, the dicarboxylic acid is adipic acid.

### BRIEF DESCRIPTION OF DRAWINGS

FIG. 1 shows a scheme of reactions according to the present invention.

### DESCRIPTION OF EMBODIMENTS

The process provides a cost competitive method of producing butadiene and/or adipic acid from a renewable feedstock. Based on the market need, the ratio of PEA/butene therefore adipic acid/butadiene can be adjusted accordingly.

### Process

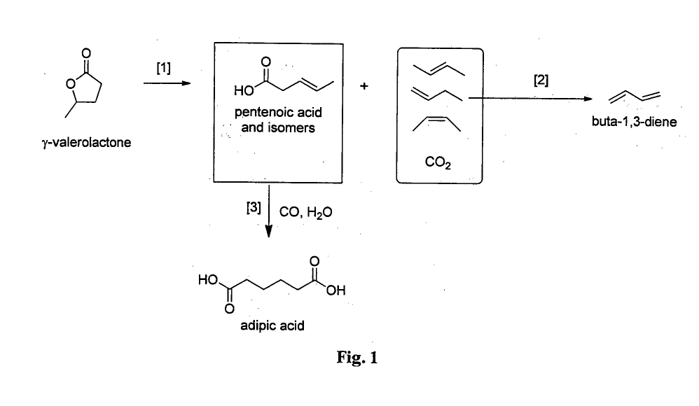

The process comprises 3 steps as show in FIG. 1:

Step 1: Pyrolyze γ-valerolactone (GVL) in a flow reactor to a mixture of pentenoic acid isomers (PEA), butene isomers, and CO₂ in the presence of an acid catalyst. Adjust reaction temperature and residence time to produce the desired ratio of PEA to butene.

Step 2: Dehydrogenate butene isomers to butadiene.

Step 3: Convert PEA isomers to adipic acid in the presence of CO, water, and a palladium catalyst. Isolate and purify adipic acid to polymerization grade product by precipitation and recrystallisation. Recycle the filtrate containing catalyst and unreacted pentenoic acid for carbonylation.

The reaction in step [1] can be carried out in the presence of an acid catalyst. A wide range of acid catalysts can be used. These include homogenous catalysts such as sulfuric acid, p-toluene sulfonic acid, and methane sulfonic acid. Heterogeneous solid acids such as alumina, zeolites, and clay are also effective. Heterogeneous solid acids are preferred.

The carbonylation reaction is commonly carried out at temperature between 80 to 120° C. and pressure ranging from 1 to 80 bars in the presence of a palladium catalyst system composed of a palladium compound, a bidentate diphosphine, and an acid. The temperature may be 80 to 100, 100 to 120 or 90 to 110° C., e.g. about 80, 85, 90, 95, 100, 105, 110, 115 or 120° C. The pressure may be 1 to 50, 1 to 20, 1 to 10, 1 to 5, 5 to 80, 10 to 80, 20 to 80, 50 to 80, 5 to 50, 5 to 20, 20 to 50 or 30 to 60 bar, e.g. about 1, 2, 3, 4, 5, 10, 15, 20, 25, 30, 35, 40, 45, 50, 55, 60, 65, 70, 75 or 80 bar. Other temperatures and pressures may also be used. Any suitable combination of the temperatures and pressures listed above may be used.

Butadiene can be obtained by direct dehydrogenation or oxidative dehydrogenation of butenes. The main reaction for direct dehydrogenation, or steam dehydrogenation is following:

C₄H₈⇄C₄H₆+H₂ (a)

This reaction is endothermic. In practice, butadiene is commonly manufactured by passing butenes over a dehydrogenation catalyst at suitable conditions of temperature and pressure. In general, the butenes are diluted with steam, both to help stabilize the temperature during the endothermic dehydrogenation reaction and to help shift the equilibrium in the desired direction by lowering the partial pressure of the product hydrogen and butadiene. On stream periods are generally short to avoid coke on the catalysts which must be regenerated at the certain period.

Along with the development of dehydrogenation of butenes, oxidative dehydrogenation of butene to butadiene has also been proposed and developed. The main reaction is following:

C₄H₈+½O₂⇄C₄H₆+H₂O (b)

This reaction is exothermic. These oxidative processes exhibit several significant advantages over the older non-oxidative processes, chief among these being that when hydrogen is removed from the system by being converted to water, the constraints of thermodynamic equilibrium are moved far to the butadiene side. For example, at 0.1 bar, the equilibrium conversions for the reaction (a) are 35% and 71% at 500° C. and 600° C., respectively, while the equilibrium conversion for the reaction (b) is essentially 100% over the complete temperature and pressure range of interest. In practice, non-oxidative commercial processes operate with 40-50% conversion with 70-90% selectivities. The oxidative processes can operate at per pass conversions of at least 70% with selectivity of 90% or higher. Another advantage of the oxidative process is that much longer on-stream periods are possible. In addition, oxygen in the vapor phase inhibits the accumulation of coke on the catalyst so that the regeneration frequency could be dramatically reduced in terms of months rather than minutes or hours.

### Chemistry

The process involves catalytic reaction of γ-valerolactone in the presence of an acid catalyst to a mixture of pentenoic acid isomers and butene isomers. This reaction can be controlled to adjust the product ratio of pentenoic acid isomers to butene isomers. The PEA isomers are fed to the carbonylation unit in the presence of water to generate adipic acid with high selectivity (FIG. 1). The butenes can be used to make butadiene via dehydrogenation.

The production of butenes from a co-feed of γ-valerolactone and water at 375 to 400° C. has been previously disclosed. The reaction was proposed to occur via the decarboxylation of pentenoic acid intermediates. The inventors note that water has a negative effect on the reaction rate and it is therefore highly preferable to exclude water in this reaction. In fact, water may inhibit acid-catalyzed surfaces as it may compete for adsorption at acid sites. It has been observed that water is necessary to minimize the extent of catalyst deactivation in the decarboxylation of PEA to produce butenes. Under the present reaction conditions, the inventors did not observe significant catalyst deactivation over a one month run. Without wishing to be bound by theory, the inventors hypothesise that ring-opening of GVL leads to a carbocationic intermediate (with the positive charge on the C4 position), which can undergo isomerisation to its carbocationic isomers. Proton abstraction then provides the various pentenoic acid isomers. The decarboxylation can occur to the carbocationic intermediate or to the pentenoic acids. The present inventors have observed that water inhibits the ring opening reaction (GVL-PEA). Previous work had suggested that water prevents catalyst deactivation, but no significant catalyst deactivation was observed in the present work. This may possibly be due to the previous work being conducted at higher temperature.

A method of producing a mixture of pentenoic acid isomers with high selectivity and without the formation of butenes by reactive distillation of GVL had been previously disclosed.

By contrast, in the current invention, a mixture of pentenoic acid isomers and butene isomers can be readily obtained in high selectivity by flowing GVL through a bed of solid acid catalyst at a temperature range between 200 and 380° C. but substantially in the absence of water. The ratio PEA to butene can be adjusted by controlling reaction variables such as temperature, space velocity, etc.

More generally, the process may comprise the step of selecting a value for at least one process variable so as to obtain a desired ratio of alkenoic acid to alkene in step (a). The process variables which may be selected include pressure, temperature and flow velocity. Thus selecting a higher reaction temperature will in general result in a higher proportion of alkene and a lower proportion of alkenoic acid, other variables being constant. Selecting a higher flow rate in general results in a higher proportion of alkenoic acid and a lower proportion of alkene, other variables being constant.

Carbonylation of 3-pentenoic acid to adipic acid can proceed with very high selectivity in the presence of a palladium catalyst derived from a palladium compound, a sterically bulky diphosphine, and an acid. A particularly effective catalyst system is derived from palladium acetate, 1,2-bis[di(t-butyl)phosphinomethyl]benzene, and methane sulfenic acid as shown below.

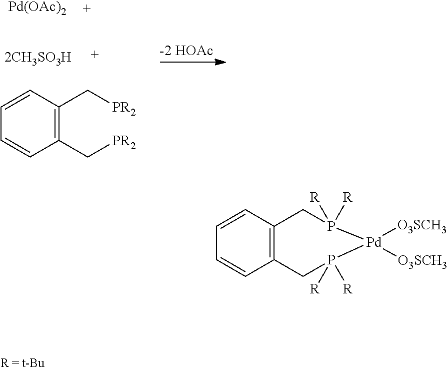

Carbonylation of a mixture of 2-pentenoic acid, 3-pentenoic acid, and 4-pentenoic acid to adipic acid with high selectivity has not been previously reported.

Conversion of adipic acid can be achieved with a process comprising:
I) Reacting at least one lactone selected from a group which consist of GVL with carbon monoxide and water in the presence of a homogeneous rhodium catalyst and at least one promoter selected from the group consisting of an iodide compound and a bromide compound; and
II) Quenching the reaction when the rate of carbon monoxide uptake declines sharply.

However, it has not previously been recognised that a mixture of pentenoic acid isomers and butene isomers could be produced via pyrolysis of γ-valerolactone in a flow reactor in the presence of an acid catalyst but absence of water.

### ILLUSTRATIVE EXAMPLES

### Example 1

### Preparation of Pentenoic Acid Isomers and Butenes Via Catalytic Reaction of γ-Valerolactone

The conversion of GVL to PEA was carried out in an up-flow fixed bed reactor (ID=6 mm) loaded with catalyst silica-alumina (Sigma Aldrich, grade 135.6 g). The reactor was seated in a tubular furnace with temperature controller. GVL (Sigma Aldrich) was fed using a HPLC pump (Lab Alliance Series I) to get the required weight-hourly space velocities (WHSV). The system pressure was controlled by a back pressure regulator at 32 bar. The reaction product was analysed by GC. The experimental results are summarized in Table 1.

TABLE 1
Reaction conversion and selectivity

| | Temper- | | GVL | Product selectivity | | |
|---|---|---|---|---|---|---|
| | ature | WHSV | conver- | % | | |
| Experiment | (° C.) | h⁻¹ | sion % | PEA | butene | others |
| 1 | 280 | 1.0 | 14.2 | 86.6 | 12.4 | 1.0 |
| 2 | 300 | 0.5 | 32.4 | 48.8 | 50.3 | 0.9 |
| 3 | 330 | 1.0 | 67.2 | 14.1 | 84.8 | 1.1 |

### Example 2

### Carbonylation of Isomeric Mixtures of Pentenoic Acids to Adipic Acid

The stainless steel 300 ml Parr reactor was charged with degassed diglyme (40 ml), degassed deionised water (5.0 ml, 278 mmol), and degassed concentrated PEA mixtures (13.6 ml, 64.3 mmol PEA isomers, distillate composition by GC: 2-PEA, 12%; 3-PEA, 20%; 4-PEA, 14%; GVL, 54%) under a stream of argon gas. The Parr reactor was evacuated and refilled with CO (2 bar). A yellow solution of catalyst consisted of palladium acetate (30.5 mg, 0.14 mmol), 1,2-bis[di(t-butyl)phosphinomethyl]benzene (108.2 mg, 0.27 mmol), and methane sulfonic acid (0.1 mL, 1.5 mmol) in diglyme (10 ml) was injected into the reactor under a stream of CO gas. After that the Parr reactor was pressurized with CO (60 bar). The reaction mixture was stirred at 1000 rpm. The Parr reactor was heated at 105° C. for 5 h. After 5 h, the reactor was cooled, vented and opened to air. A yellow reaction mixture was obtained which was placed in the fridge to crystallize out the adipic acid. The crude adipic acid was filtered, washed with ethyl acetate and dried under vacuum at 60° C. to give 1.999 g of adipic acid crystals (13.7 mmol). Analysis of the reaction mixture by NMR showed adipic acid as the only product.

1. A process for the production of a diene comprising the steps of:
(a) heating a lactone in the presence of a first catalyst system and substantially in the absence of water to produce an alkene and carbon dioxide; and
(b) contacting the alkene with a second catalyst system to produce an alkyldiene.

2. The process of claim 1 additionally comprising the step of drying the lactone prior to step (a).

3. The process of claim 1 or claim 2 wherein step (a) is conducted in a flow reactor.

4. The process of any one of claims 1 to 3 wherein step (a) is carried out at a temperature at or above the normal boiling point of the lactone.

5. The process of any one of claims 1 to 4 wherein step (a) is carried out at a pressure of between about 0.5 bar and 50 bar.

6. The process of any one of claims 1 to 5 wherein the first catalyst system comprises an acidic catalyst.

7. The process of any one of claims 1 to 6 wherein the first catalyst system is a heterogeneous solid catalyst.

8. The process of any one of claims 1 to 7 wherein step (b) comprises:
i. direct dehydrogenation of the alkene to produce the alkyldiene or
ii. oxidative dehydrogenation of the alkene to produce the alkyldiene.

9. The process of any one of claims 1 to 8 wherein a portion of the lactone is unreacted after step (a), whereby step (a) produces a reaction composition comprising unreacted lactone and the alkene.

10. The process of claim 9 further comprising the steps of:
(a1) separating part, or substantially all, of the unreacted lactone from the alkene; and
(a2) recycling the separated unreacted lactone from step (a1) to step (a).

11. The process of any one of claims 1 to 10 wherein a portion of the alkene is unreacted after step (b) whereby a reaction composition comprising unreacted alkene and alkyldiene is produced in step (b).

12. The process of claim 11 further comprising the step of:
(b1) separating part or substantially all of the unreacted alkene from the alkyldiene; and
(b2) recycling the separated unreacted alkene from step (b1) to step (b).

13. The process of any one of claims 1 to 12 wherein step (a) also produces an alkenoic acid.

14. The process of claim 13 further comprising the step of:
(c) contacting the alkenoic acid with carbon monoxide, water and a third catalyst system to produce a reaction composition comprising dicarboxylic acid.

15. The process of claim 14 wherein step (c) is carried out substantially in the absence of oxygen.

16. The process of claim 14 or claim 15 wherein step (c) is carried out at a temperature of between about 50° C. and about 150° C.

17. The process of any one of claims 14 to 16 wherein step (c) is carried out at a pressure of between about 1 bar and about 80 bar.

18. The process of any one of claims 14 to 17 wherein, the third catalyst system comprises a palladium catalyst.

19. The process of claim 18 wherein the palladium catalyst has formula (I): 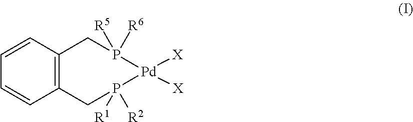
wherein X is a ligand and each of R¹, R², R⁵ and R⁶ is independently an optionally substituted hydrocarbon group.

20. The process of claim 19 comprising the step of preparing the palladium catalyst by combining a palladium compound, a bidentate diphosphine and an acid.

21. The process of claim 20 wherein the step of preparing the palladium catalyst is conducted in situ.

22. The process of claim 20 or claim 21 wherein the palladium compound is selected from the group consisting of palladium carboxylates and palladium(0) compounds.

23. The process of any one of claims 20 to 22 wherein the acid is selected from the group consisting of sulfonic acids, sulphuric acids, phosphorous acids and carboxylic acids.

24. The process of any one of claims 20 to 23 wherein the bidentate diphosphine for the palladium catalyst preparation is 1,2-bis[di(t-butyl)phosphinomethyl]benzene.

25. The process of any one of claims 14 to 24 wherein a portion of the lactone is unreacted after step (a), producing a reaction composition comprising unreacted lactone, alkenoic acid and alkene, and part or substantially all of the unreacted lactone is present in step (c), producing a reaction composition comprising unreacted lactone and dicarboxylic acid.

26. The process of claim 25 further comprising the steps of:
(c1) separating part or substantially all the unreacted lactone from the dicarboxylic acid; and
(c2) recycling the separated unreacted lactone from step (c1) to step (a).

27. The process of any one of claims 14 to 26 wherein a portion of the alkenoic acid is unreacted after step (c), producing a reaction composition comprising unreacted alkenoic acid and dicarboxylic acid from step (c).

28. The process of claim 27 further comprising the steps of:
(c3) separating part or substantially all of the unreacted alkenoic acid from the dicarboxylic acid; and
(c4) recycling the separated unreacted alkenoic acid from step (c3) to step (c).

29. The process of any one of claims 14 to 28 further comprising the step of:
(c5) separating the dicarboxylic acid from the remainder of the reaction composition by reducing the temperature to produce a first portion comprising the dicarboxylic acid and substantially none of the third catalyst system, and a second portion comprising the third catalyst system.

30. The process of claim 29 additionally comprising the step of:
(c6) recycling the second portion, comprising the third catalyst system, to step (c).

31. The process of any one of claims 1 to 30 wherein the lactone is γ-valerolactone, whereby the alkene is butene and the alkyldiene is 1,3-butadiene.

32. The process of claim 31 comprising the step of preparing the γ-valerolactone by hydrogenation of levulinic acid.

33. The process of claim 32 comprising preparing the levulinic acid by acid catalysed hydrolysis of cellulose.

34. The process of claim 33 comprising preparing the cellulose by cracking lignocellulose.
